(19) United States
(12) Patent Application Publication
(10) Pub. No.: US 2008/0058639 A1
(43) Pub. Date: Mar. 6, 2008

(54) MEDICAL NETWORK SYSTEM, MEDICAL IMAGING APPARATUS, MEDICAL IMAGE PROCESSOR, AND MEDICAL IMAGE PROCESSING METHOD

(75) Inventors: Hideyuki SAKAIDA, Tokyo (JP); Naoki MOCHIZUKI, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 11/845,204

(22) Filed: Aug. 27, 2007

(30) Foreign Application Priority Data
Aug. 29, 2006 (JP) 2006-231603

(51) Int. Cl. A61B 6/03; G06K 9/20

(52) U.S. Cl. 600/425; 382/131

(57) ABSTRACT

A medical network system includes a computed tomography apparatus for imaging a body to acquire plural tomographic medical images as data files. An image server processes an image group of the tomographic medical images transferred from the computed tomography apparatus by network transmission. In the computed tomography apparatus, an imaging number of a sequence of imaging of each of the tomographic medical images is generated. An image end flag is generated, and represents a final tomographic medical image acquired finally in the image group. The image server includes CPU for checking the imaging number and the image end flag and for verifying completion of transfer of the tomographic medical images in the image group. The image server includes a site recognition unit for recognizing a site of the body recorded in each of the tomographic medical images by image recognition, to create site information.

### BACKGROUND OF THE INVENTION

1. Field of the Invention

The present invention relates to a medical network system, medical imaging apparatus, medical image processor, and medical image processing method. More particularly, the present invention relates to a medical network system in which transfer of medical images can be checked reliably even with a considerably great amount of image data, and a medical imaging apparatus, medical image processor, and medical image processing method.

2. Description Related to the Prior Art

Medical imaging apparatuses of various modalities are used in a hospital or other medical facilities, for example, CR (Computed Radiography) apparatus, CT (computed tomography) apparatus, MRI (magnetic resonance imaging) apparatus, PET (positron emission tomography) apparatus or the like. Medical images are output by imaging apparatuses, and used for diagnosis and treatment of a patient as important elements in the field of the medicine.

In general, data of medical images are preserved in the medical facilities for a number of years as a post-stroke period after the diagnosis and treatment. If films or other physical media are used for storing image data of a huge amount, problems are serious in the maintenance of a space of preservation, indexing and management. To solve such problems, JP-A 2002-288344 discloses a system in which electronic data of medical images are used and stored in an image server. The use of the image server in medical facilities is advantageous in a reduced space in comparison with a storing space of physical media. Also, it is possible to raise efficiency in the operation in the medical facilities by automation of the data management of the image server.

Imaging apparatuses, such as a CT apparatus and MRI apparatus, acquire a plurality of medical images in one diagnosis for various positions of imaging. It is preferable to store plural medical images in an image server or image manager in a sorted manner for diagnoses. However, the amount of image data of medical images is considerably great so that very long time is required for transfer to the image server or image manager. If a radiologist wishes to process transferred medical images, the efficiency of processing is low because of waiting time taken before the transfer of the all the medical images.

An example of transfer of medical images is transfer of those to the image server discretely one after another. An advantage of this lies in a short time for each of the medical images. As soon as the transfer of one medical image is completed, the medical image can be processed for various purposes. However, there is no confirmation of the completion of all the medical images in the image server. If failure occurs in the transfer of any one of the medical images, missing in the preservation is created.

JP-A 2002-288344 discloses medical image management in which attribute information is assigned to each of medical images and includes a serial number according to imaging sequence and the total number of images input at the time of reservation of diagnosis. It is possible in this image management that the image server easily determines whether the transfer of all medical images of one diagnosis is completed by checking the attribute information even after discrete transmission of medical images one after another.

There arises a problem in that the total number of medical images input at the time of reservation of diagnosis is different from the number of actually acquired medical images. It has been recently conceived to prevent repeated imaging in the case of failure of imaging of a particular site or lesion. According to high performance of imaging of any of various modalities, it is likely that a greater number of medical images are recorded than the number of medical images predetermined by request information, for example by imaging a comparatively large region of a site, or imaging at a small slice thickness in operation of a radiologist. However, it is impossible in JP-A 2002-288344 to discern the completion of transfer of all the medical images, because of a temporary difference between the total number of medical images input at the time of reservation of diagnosis and the number of actually acquired medical images.

### SUMMARY OF THE INVENTION

In view of the foregoing problems, an object of the present invention is to provide a medical network system in which transfer of medical images can be checked reliably even with a considerably great amount of image data, and a medical imaging apparatus, medical image processor, and medical image processing method.

In order to achieve the above and other objects and advantages of this invention, a medical network system is provided, including a medical imaging apparatus for imaging a body to acquire plural medical images of a consecutive series, and a medical image processor for processing an image group of the medical images transferred from the medical imaging apparatus by network transmission. In the medical network system, the medical imaging apparatus includes a sequence information generator for generating sequence information of a sequence of imaging of each of the medical images. An image end information generator generates image end information representing a final medical image acquired finally in the image group. A transmitter transfers the medical images to the medical image processor. The medical image processor includes a checker for checking the sequence information and the image end information and for verifying completion of transfer of the medical images in the image group.

The medical image processor performs a first task related to the medical images discretely from one another, and then a second task related to the medical images in total after the checker verifies the completion of the transfer of the medical images.

The medical image processor further includes a site recognition unit for recognizing a site of the body recorded in each of the medical images by image recognition, to create site information of the medical images.

The site recognition unit recognizes the medical images discretely from one another, and recognizes the medical images in total after the checker verifies the completion of the transfer of the medical images. In the discrete recognition, the site information of the medical images is determined in provisional determination by image analysis of the medical images. In the recognition in total, the site information is checked, to determine new site information of the medical images in final determination by considering an error in recognition.

The checker identifies the final medical image according to the image end information, and determines the completion of the transfer of the medical images in the image group when a number of the final medical image coincides with a number of the medical images by referring to the sequence information of the final medical image.

The sequence information is metadata assigned to respectively the medical images, and the image end information is metadata assigned to the final medical image.

The sequence information generator sequentially assigns the sequence information to the medical images being acquired, and the transmitter transfers the medical images to the medical image processor sequentially after assignment of the sequence information.

The medical images are tomographic.

Also, a medical imaging apparatus for imaging a body is provided, to acquire an image group constituted by plural medical images of a consecutive series. A sequence information generator generates sequence information of a sequence of imaging of each of the medical images. An image end information generator generates image end information representing a final medical image acquired finally in the image group.

Furthermore, a transmitter transfers the medical images, the sequence information and the image end information. The sequence information generator sequentially assigns the sequence information to the medical images being acquired, and the transmitter transfers the medical images sequentially after assignment of the sequence information. The sequence information and the image end information are used for verifying completion of transfer of the medical images in the image group after transfer of the medical images.

Also, a medical image processor for processing an image group of medical images transferred by network transmission is provided, the medical images being acquired in a consecutive series. A checker is supplied with sequence information of a sequence of imaging of each of the medical images of the image group and image end information representing a final medical image acquired finally in the image group, for verifying completion of transfer of the medical images in the image group by checking the sequence information and the image end information.

Also, a medical image processing method of image processing of an image group of plural medical images received discretely by network transmission is provided, the medical images being acquired in a consecutive series. In the medical image processing method, the medical images are recognized discretely from one another by image recognition. Completion of transfer of the medical images in the image group is verified according to sequence information of a sequence of imaging of each of the medical images, and image end information representing a final medical image acquired finally in the image group. The medical images in total are recognized by image recognition after the completion of the transfer of the medical images is verified.

Also, a computer executable program for medical image managing is provided, in combination with a medical imaging apparatus for outputting plural medical images which constitute an image group, wherein an information generator outputs attribute data of the medical image, and a communication interface transfers the medical images and the attribute data by network transmission. The computer executable program includes a managing program code for managing medical images transferred by the communication interface. A checking program code is for checking the attribute data and for verifying completion of transfer of the medical images in the image group by the communication interface.

Furthermore, there is a program code for recognizing the medical images discretely from one another by image recognition. A program code is for recognizing the medical images in total by image recognition after the completion of the transfer of the medical images is verified.

Also, a user interface for medical image managing is provided, in combination with a medical imaging apparatus for outputting plural medical images which constitute an image group, wherein an information generator outputs attribute data of the medical image, and a communication interface transfers the medical images and the attribute data by network transmission. The user interface includes a managing region for managing medical images transferred by the communication interface. A checking region is for checking the attribute data and for verifying completion of transfer of the medical images in the image group by the communication interface.

Furthermore, there is a region for recognizing the medical images discretely from one another by image recognition. A region is for recognizing the medical images in total by image recognition after the completion of the transfer of the medical images is verified.

Accordingly, transfer of medical images can be checked reliably even with a considerably great amount of image data, because the sequence information of the medical images and the image end information of the final medical images are evaluated in combination.

### BRIEF DESCRIPTION OF THE DRAWINGS

The above objects and advantages of the present invention will become more apparent from the following detailed description when read in connection with the accompanying drawings, in which:

FIG. 1 is a block diagram schematically illustrating a medical network system;

FIG. 2 is a perspective view illustrating a computed tomography apparatus;

FIG. 3 is an explanatory view illustrating a relationship between volume data and tomographic medical images;

FIG. 4 is a block diagram schematically illustrating the computed tomography apparatus;

FIG. 5 is a block diagram schematically illustrating the image server;

FIG. 6 is a flow chart illustrating operation of the computed tomography apparatus; and

FIG. 7 is a flow chart illustrating operation of the image server.

### DETAILED DESCRIPTION OF THE PREFERRED EMBODIMENT(S) OF THE PRESENT INVENTION

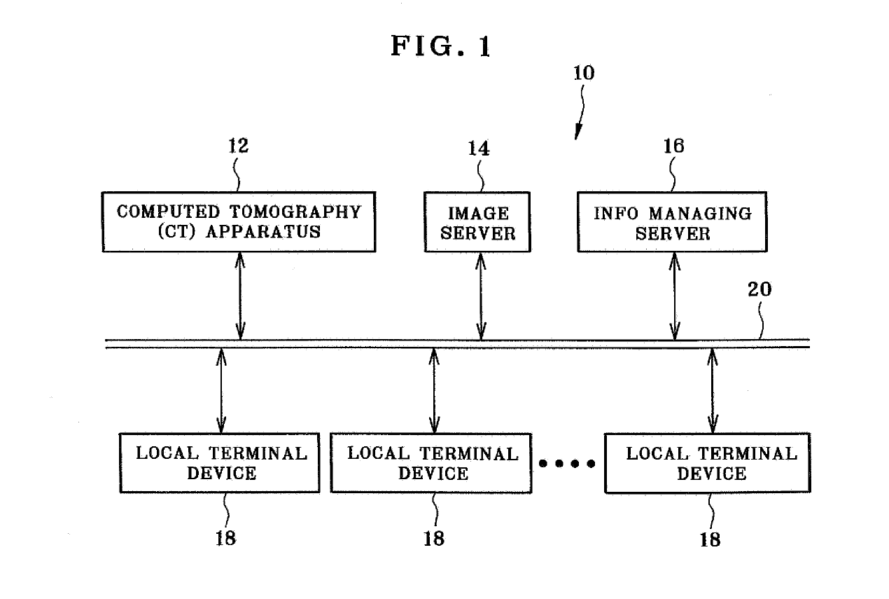

In FIG. 1, a construction of a medical network system as architecture 10, for example PACS (Picture Archiving and Communications System) for use in a hospital or other clinical facilities is schematically illustrated. The medical network system 10 includes a computed tomography (CT) apparatus 12 or medical imaging apparatus, an image server 14 as image processor or image manager, an information managing server 16, and plural local terminal devices 18 for physicians or staff. The computed tomography apparatus 12 acquires a tomographic medical image of a body of a patient as a medical image. The image server 14 stores tomographic medical images or data files retrieved by the computed tomography apparatus 12. The information managing server 16 manages information of various items used in the medical facilities. The local terminal devices 18 are used by physicians for consultation or treatment. A local area network (LAN) 20 in the medical facilities connects those components with one another.

The medical network system 10 manages electronic data including various medical data, medical image data and the like generated in medical facilities, for the purpose of reducing storage spaces for medical notebooks, medical film and the like in the medical facilities. Also, the medical network system 10 makes it possible at the local terminal devices 18 to read various data, medical image data and the like with great ease, to raise efficiency in operation of the medical facilities.

The image server 14 is a server of the PACS. Various images are stored in the image server 14, including CT images from the computed tomography apparatus 12, medical images provided by other medical facilities through the network or storage medium, reference images for use with comparison of illness or injury, and the like. Medical images stored in the image server 14 are read by the local terminal devices 18 when required, and used for diagnostic observation by physicians or for explanation to patients. Images of various modalities are included in medical images herein, for example CT images obtained by the computed tomography apparatus 12, and CR (Computed Radiography) images and MRI (Magnetic Resonance Imaging) images. Note that reference images can be any type of images other than those acquired by imaging, for example a graphically produced image manually input by use of an image editor, CAD and the like.

Examples of the information managing server 16 include an HIS (hospital information system) server, an RIS (radiology information system) server and the like. The information managing server 16 manages various kinds of information for each of patients, the information including personal information, consultation information, diagnosis information, accounting information and the like. The personal information may include a name, ID information, home address, age, sex, family information, past medical history, and allergy history.

Examples of the consultation information include a date of consultation, hospital department, name of illness or injury, result of consultation, period of treatment, type and dose of drug, drug store, and the like. The period of treatment is a period during which a patient repeatedly comes to the medical facilities for one illness or injury. Examples of the diagnosis information include a date, instrument, method and site of the diagnosis, any of which is combined with medical images. Examples of information of the method of the diagnosis are a front direction and lateral direction of a body of a patient, and use or no use of tomography agent. Examples of the sites include a head, chest, abdomen, pelvic bone, leg, head/neck region or chest/abdominal region. Examples of the accounting information include charge for the consultation, charge for drug, charge for diagnosis, and status of health insurance.

A plurality of the local terminal devices 18 are installed in clinical facilities, for example one for each of the treatment rooms or one for each of hospital departments, and accessed for inputting information. The local terminal devices 18 are operated by a physician observing a patient for consultation, and accessed for inputting information. Also, the local terminal devices 18 are used when a physician explains a result of the diagnosis to a patient, and displays a tomographic medical image from the computed tomography apparatus 12, information read from the information managing server 16 and the like, as a clinical support. Examples of the local terminal devices 18 are a personal computer, workstation, or other electronic devices well known in the art.

A reservation list for the computed tomography apparatus 12 is formatted and stored in the information managing server 16. If a physician finds requirement of imaging with the computed tomography apparatus 12, he or she accesses the information managing server 16 by use of the local terminal device 18 to view the reservation list of the computed tomography apparatus 12. The physician inputs request information for an available date and time of the computed tomography apparatus 12, and reserves the computed tomography apparatus 12 in the reservation list for the inspection. The request information is transferred by the information managing server 16 to the local terminal device 18 in the radiology department, the computed tomography apparatus 12 and other devices. An operator or radiologist of the computed tomography apparatus 12 views the request information by accessing the local terminal device 18 or the computed tomography apparatus 12, to form a CT image according to the request information.

The information managing server 16 manages information of patients, and a status of the use of the computed tomography apparatus 12 to prevent double booking of the diagnosis in the same time zones. Note that the request information includes personal information, staff information, method information, site information and the like. The staff information includes a name of a physician instructing the inspection, his or her department, and telephone number or other contact information, and the like.

In FIG. 1, a plurality of the local terminal devices 18 are arranged. However, a single local terminal device 18 may be used in the medical network system 10. Also, a plurality of the computed tomography apparatus 12, a plurality of the image server 14, a plurality of the information managing server 16 may be combined in the medical network system 10.

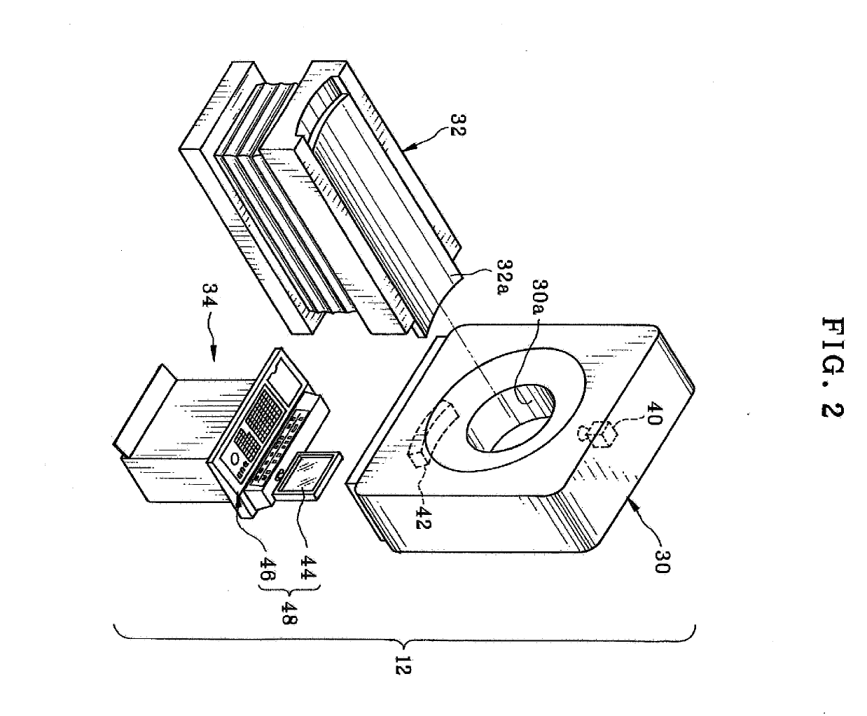

In FIG. 2, appearance of the computed tomography apparatus 12 is illustrated. The computed tomography apparatus 12 includes a CT gantry 30, a table assembly 32, and a controller 34 or control machine. The CT gantry 30 includes an X ray radiation source 40 and an X ray detector 42. The X ray radiation source 40 applies X rays to a body of a patient. The X ray detector 42 detects X rays transmitted through the patient. A diagnostic gantry opening 30a is formed in the CT gantry 30. The table assembly 32 supports the body and slides it into the gantry opening 30a. The controller 34 controls the CT gantry 30 and the table assembly 32. There is a scanner room in which the CT gantry 30 and the table assembly 32 are installed. A control room is separated from the scanner room, and contains the controller 34 installed therein.

The X ray radiation source 40 and the X ray detector 42 are opposed to one another beside the gantry opening 30a, and are positioned on the CT gantry 30 in a concentric manner about an axis of the CT gantry 30. While a body lies in the gantry opening 30a, the CT gantry 30 rotates the X ray radiation source 40 and the X ray detector 42 about the axis, and causes the X ray radiation source 40 to emit X rays to the body. Projection data of various directions are retrieved.

A table board 32a is included in the table assembly 32 on which a body of a patient is placed. The table board 32a is moved in the table assembly 32 vertically between a low position and a high position, and when in the low position, is lower than the gantry opening 30a to allow a patient to get on or get off easily, and when in the high position, is ready and positioned as high as the gantry opening 30a. The table assembly 32 slides the table board 32a in the high position horizontally to enter the gantry opening 30a.

A user interface or console panel 48 is provided in the controller 34, and includes a monitor or display panel 44 and an input interface 46. The display panel 44 displays various screen patterns of menus for inputs. The input interface 46 has a keyboard, various switches and the like. The controller 34 is connected to the CT gantry 30 and the table assembly 32 by a cable (not shown). The controller 34 controls the CT gantry 30 and the table assembly 32 in response to command signals input with the input interface 46 by an operator or radiologist.

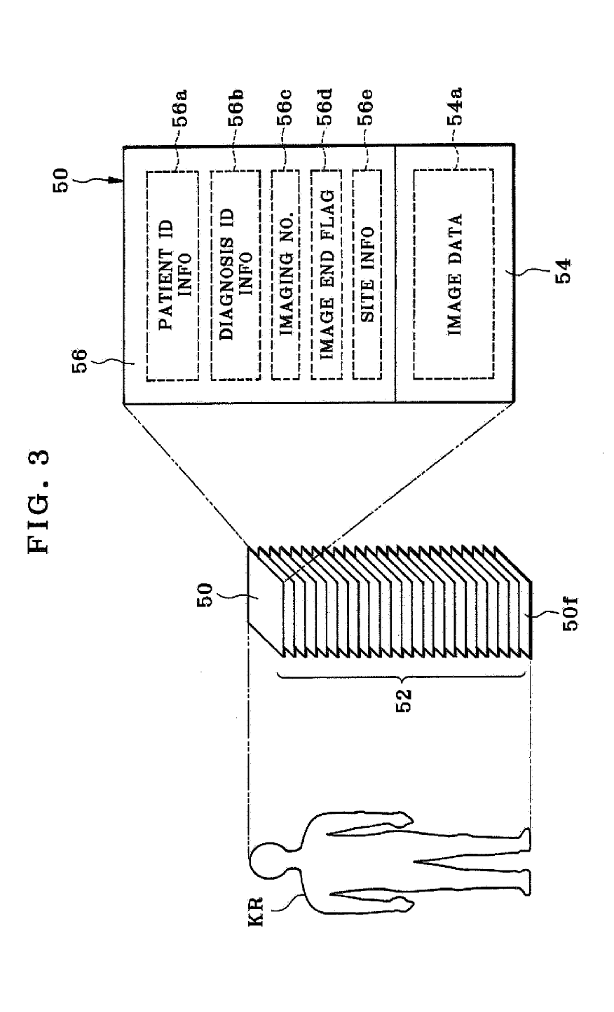

The computed tomography apparatus 12 images the body of the patient KR according to the request information. In FIG. 3, tomographic medical images as data files 50 of plural image frames are acquired by the computed tomography apparatus 12 according to the slice thickness and the like. Each of the tomographic medical images 50 is transferred to the image server 14, and stored in the image server 14 in a grouped manner for diagnoses. A term of volume data or image group 52 is used for a group of the tomographic medical images 50 of one diagnosis.

In FIG. 3, each data file of the tomographic medical images 50 has an image region 54 and a tag region 56 for attribute data. Image data 54a is recorded in the image region 54. Various attribute data are recorded in the tag region 56 of the tomographic medical images 50 as metadata, including patient ID information 56a, diagnosis ID information 56b, imaging number 56c as sequence information, an image end flag 56d, and site information 56e of a site or tissue. The patient ID information 56a is used for discerning the patient KR from which the tomographic medical images 50 are obtained. The diagnosis ID information 56b is a number discretely assigned to the diagnosis. The diagnosis ID information 56b is used for discernment according to the respective diagnosis, and for management of the tomographic medical images 50 in the volume data 52.

The imaging number 56c is according to the sequence of the tomographic medical images 50 included in the volume data 52. The image end flag 56d is a flag for discriminating a final tomographic medical image 50f in the volume data 52 recorded finally. The image end flag 56d is a one-bit flag of which the value is set as 1 (one) for the final tomographic medical images 50 in the volume data 52 recorded finally, and set as 0 (zero) for any remaining one of the tomographic medical images 50 in the volume data 52 recorded before the final recording. Note that the definition of the image end flag 56d may be the contrary to the present example. The number of the bits of the image end flag 56d may be more than one (1).

The site information 56e is information of a site recorded in the tomographic medical images 50. Various attribute data or metadata are recorded at the same time as the image data 54a is formed in obtaining the tomographic medical images 50. Metadata of the tag region 56 are not limited to those examples, and may be any attribute information with which the tomographic medical images 50 can be discerned. An example of a file format of medical images having the tag region 56 is DICOM (Digital Imaging and Communication in Medicine) format.

For use with the DICOM format of the tomographic medical images 50, an example of the patient ID information 56a is Patient ID (0010,0010) prepared as a public tag. An example of the diagnosis ID information 56b is Study ID (0020,0010) prepared as a public tag. Examples of the imaging number 56c are Acquisition Number (0020,0012), Instance Number (0020,0013) and the like prepared as a public tag. An example of the image end flag 56d is Image Comments (0020,4000) prepared as a public tag. An example of the site information 56e is Body Part Examined (0018,0015) prepared as a public tag. Also, the image end flag 56d may be associated with a private tag determined by a user as desired.

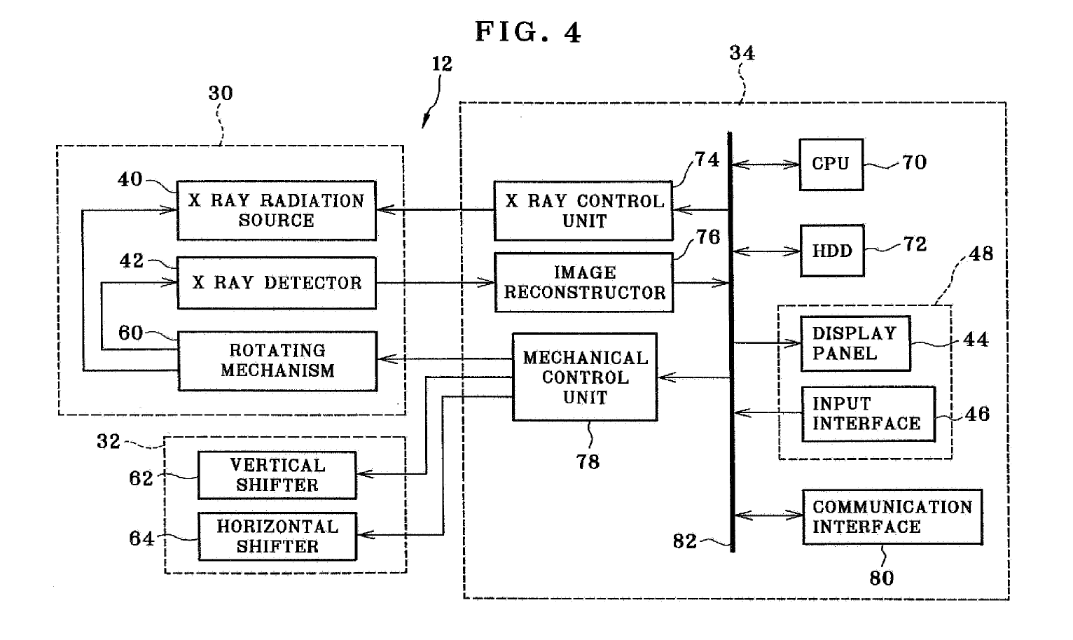

In FIG. 4, circuits in the computed tomography apparatus 12 are illustrated schematically. A rotating mechanism 60 is disposed in the CT gantry 30 together with the X ray radiation source 40 and the X ray detector 42. A vertical shifter 62 is included in the table assembly 32 for shifting the table board 32a vertically. A horizontal shifter 64 is included in the table assembly 32 for sliding the table board 32a horizontally. For any of those, a motor, gears and other known mechanical elements are used.

The controller 34 includes the console panel 48, a CPU 70 for sequence information generation and image end information generation in the image manager, a hard disk drive (HDD) 72 as data storage, an X ray control unit 74, an image reconstructor 76, a mechanical control unit 78, and a communication interface or transmitter 80. There is a data bus 82 for interconnecting those elements. The hard disk drive 72 stores various programs for control of the computed tomography apparatus 12. The CPU 70 reads the programs from the hard disk drive 72 and controls elements of the computed tomography apparatus 12 by sequential tasks. The CPU 70 is responsive to command signals input manually with the input interface 46 by an operator or radiologist, and performs various tasks of processing. The CPU 70 causes the display panel 44 to display information of a screen pattern of a menu.

The X ray control unit 74 is connected with the X ray radiation source 40. The X ray control unit 74 transmits a control signal to the X ray radiation source 40 in response to a command signal from the rotating mechanism 60, and controls time of radiation and dose of radioactive energy of the X ray radiation source 40. The image reconstructor 76 is connected with the X ray detector 42. The image reconstructor 76 forms the tomographic medical images 50 by reconstructing the image data 54a according to projection data of various directions as output of the X ray detector 42. An example of method of the reconstruction of projection data in the image reconstructor 76 is a back projection method or the like known in the art.

The mechanical control unit 78 is connected with the rotating mechanism 60 and the vertical and horizontal shifters 62 and 64. Drivers are included in the mechanical control unit 78 and associated with respectively the rotating mechanism 60 and the vertical and horizontal shifters 62 and 64. The mechanical control unit 78 is responsive to command signals from the CPU 70, and sends a drive signal to drive the rotating mechanism 60 and the vertical and horizontal shifters 62 and 64.

The communication interface 80 connects the controller 34 to the LAN 20 of the medical facilities. An example of the communication interface 80 is Ethernet (trade name) interface or the like selected according to the specifics of the LAN 20.

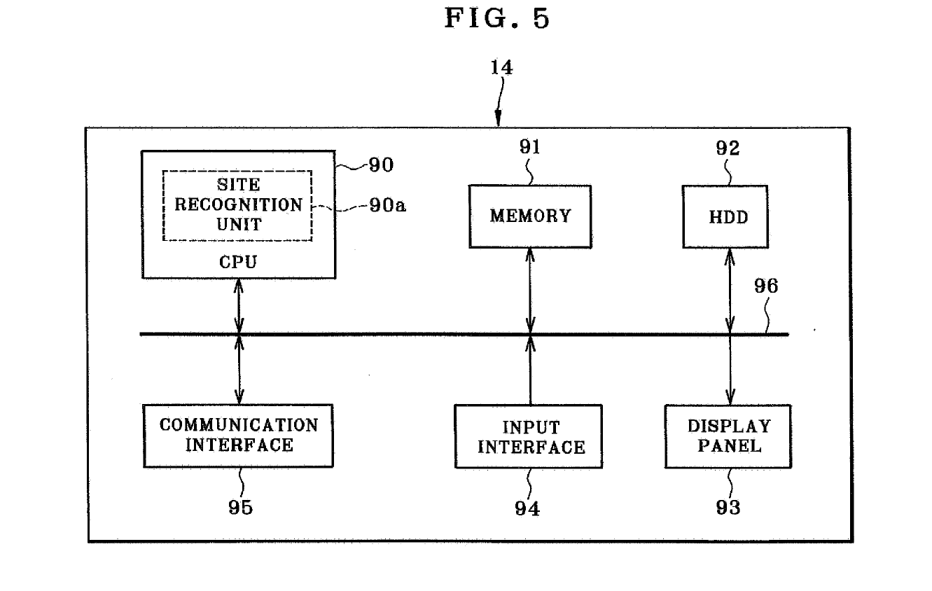

In FIG. 5, the construction of the image server 14 is schematically illustrated. The image server 14 for managing is constituted by a well-known personal computer, work station or the like, and includes a CPU 90 as checker in the image manager, a memory 91, a hard disk drive or HDD 92 as data storage, a monitor or display panel 93, an input interface 94 and a communication interface 95. A data bus 96 interconnects those elements.

The hard disk drive 92 stores managing programs according to the medical network system 10. The CPU 90 reads the programs from the hard disk drive 92, runs them by means of the memory 91, and performs tasks sequentially to control the image server 14.

Also, the hard disk drive 92 stores data files or tomographic medical images 50 retrieved by the computed tomography apparatus 12, reference images or the like. When images are input to the CPU 90 through the communication interface 95, the CPU 90 writes the images to the hard disk drive 92 at locations in a predetermined region. Also, the CPU 90 reads images from the hard disk drive 92 according to command signals from any one of the local terminal devices 18, and transfers images to the relevant one of the local terminal devices 18.

In the above embodiment, the hard disk drive 92 is commonly used for storing the programs and images. However, one storage medium or HDD may be added for storing images. In the above embodiment, the hard disk drive 92 is a built-in device. However, data storage medium in the invention may be an external storage medium, for example, DVD-ROM and CD-ROM.

The display panel 93 displays any of various screen patterns of menus according to processing of programs in the CPU 90. Examples of the display panel 93 are liquid crystal display panel, CRT display panel, and other devices known in the art. Examples of the input interface 94 include a keyboard, keypad, mouse and the like. The display panel 93 and the input interface 94 are accessed by staff of management in the medical facilities for renewal of the programs in the hard disk drive 92, and checking the status of storing images in the hard disk drive 92. The communication interface 95 connects the image server 14 to the LAN 20 in the medical facilities. An example of the communication interface 95 is an Ethernet (trade name) interface or the like selected according to the specifics of the LAN 20.

A site recognition unit 90a as image recognition unit is incorporated in the CPU 90. The tomographic medical images 50 are input to the site recognition unit 90a, and analyzed in the image recognition to recognize a site recorded in any one of the tomographic medical images 50. The site recognition unit 90a writes the site information 56e of the site to the tag region 56 of the tomographic medical images 50. An example of method of the image recognition in the site recognition unit 90a is to determine characteristic values of an image according to the CT values of pixels, and to match the characteristic values by comparison with predetermined characteristic values of sites.

There has been a recent tendency in that a large region in a body of a patient, for example the whole body, are targeted for imaging with high precision and preventing failure and retrial, together with the higher performance of the computed tomography apparatus 12 and the increase in capacity of the image server 14. However, the region of the imaging may be much larger than required for a physician's diagnostic observation of the image. There are shortcomings in long waiting time of the staff for the transfer due to unnecessary portion of the tomographic medical images 50, and in much work for searching an intended portion of the tomographic medical images 50 with important sites for the diagnosis among all of the tomographic medical images 50.

To prevent such difficulty, the image server 14, responsive to reception of the volume data 52 from the computed tomography apparatus 12, sends the volume data 52 to the site recognition unit 90a. Image recognition is carried out to recognize sites recorded in the tomographic medical images 50 of the volume data 52. Only a portion of the site information, according to a request from one of the local terminal devices 18, is transmitted. This is effective in preventing occurrence of extremely long waiting time of a physician or the like.

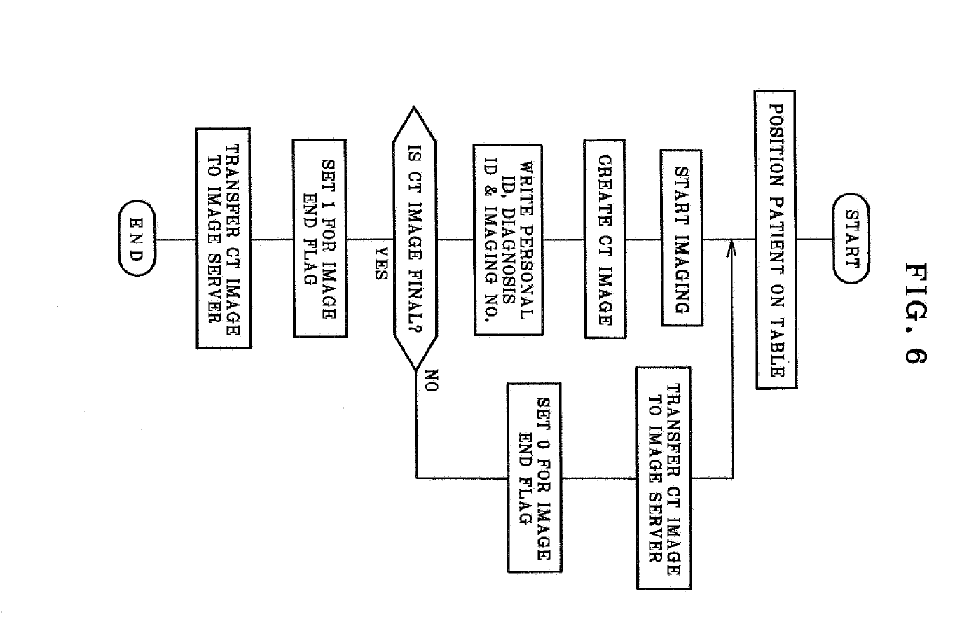

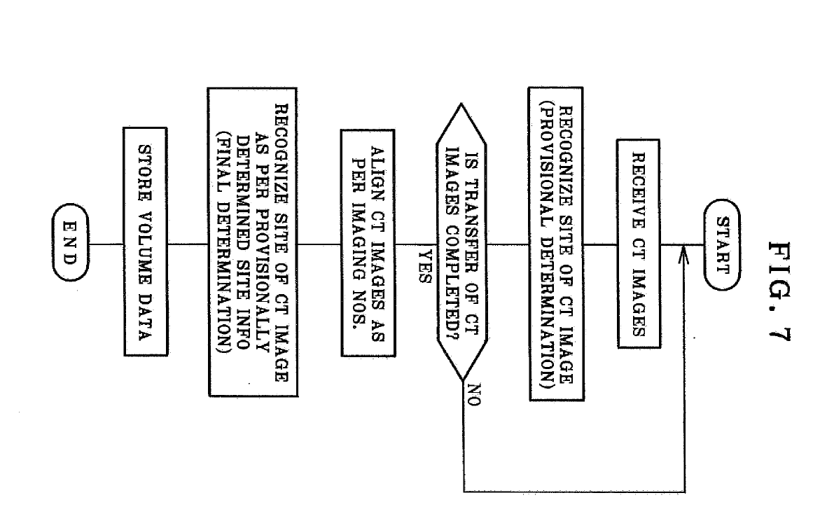

The operation of the medical network system 10 is described by referring to the flows in FIGS. 6 and 7. In FIG. 6, operation of the computed tomography apparatus 12 is illustrated. In FIG. 7, operation of the image server 14 is illustrated. If a physician finds requirement of imaging with the computed tomography apparatus 12, he or she accesses the information managing server 16 by use of the local terminal devices 18 to view a reservation list for reservation of the computed tomography apparatus 12. The physician inputs request information at a location in the reservation list for an available date and time of the computed tomography apparatus 12, and reserves the computed tomography apparatus 12 for the diagnosis. The request information is transferred by the information managing server 16 to the local terminal devices 18, the computed tomography apparatus 12 and other devices in the radiology department of the hospital. An operator or radiologist of the computed tomography apparatus 12 views the request information by accessing the local terminal device 18 or the computed tomography apparatus 12, to image a body of the patient KR according to the request information.

An operator or radiologist of the computed tomography apparatus 12 places the patient KR on the table board 32a of the table assembly 32, and keeps the patient KR positioned by a belt, protector or the like. Then the operator operates the input interface 46 to drive the vertical shifter 62, to set the table board 32a as high as the gantry opening 30a. When the height of the table board 32a is adjusted, the operator operates the input interface 46 to determine a region of imaging and the like, and sends a signal to the controller 34 to start imaging.

The CPU 70 of the controller 34, responsive to a command signal for a start of imaging by an operator or radiologist, drives the horizontal shifter 64 to move the patient KR to a position according to an imaging region. After this, the CPU 70 starts the X ray radiation source 40 to emit X rays. Simultaneously, the CPU 70 drives the rotating mechanism 60, to cause the X ray radiation source 40 and the X ray detector 42 to rotate about an axis of the body of the patient KR. The X ray radiation source 40 and the X ray detector 42 retrieve projection data in various directions in a period of one rotation, to output the projection data to the image reconstructor 76.

The image reconstructor 76 provided with projection data reconstructs the image data 54a to create the tomographic medical images 50, which are written to the hard disk drive 72. The CPU 70 responsive to the creation of the tomographic medical images 50 accesses the hard disk drive 72, and writes the patient ID information 56a and the diagnosis ID information 56b to the tag region 56 of the created tomographic medical image 50 according the request information. Also, the CPU 70 counts the tomographic medical images 50 output by the image reconstructor 76 during the diagnosis, and obtains a count number as the imaging number 56c, which is written to the tag region 56.

The CPU 70 after recording the attribute data or metadata checks whether the created tomographic medical image 50 is the final tomographic medical image 50f in the volume data 52 according to the imaging region, slice thickness and other information of the setting. If the created tomographic medical image 50 is determined not final, then the CPU 70 sets 0 (zero) for the image end flag 56d of the created tomographic medical image 50. The tomographic medical image 50 is transferred by the CPU 70 to the image server 14 after setting the image end flag 56d. The CPU 70 drives the horizontal shifter 64 to move the table board 32a at a distance according to the slice thickness while the tomographic medical image 50 is transferred, and starts acquisition of a second one of the tomographic medical images 50. A data stream of the volume data 52 is transferred. Thus, the transfer of the tomographic medical images 50 is made efficient by sequential transfer of the tomographic medical images 50 from the computed tomography apparatus 12 simultaneously with retrieval of the second tomographic medical image 50.

If the created tomographic medical image 50 is found the final tomographic medical image 50f, the CPU 70 sets a value one (1) to the image end flag 56d of the tomographic medical images 50. Then the CPU 70 stops application of X rays from the X ray radiation source 40 and stops driving the rotating mechanism 60. The table board 32a is moved back to the initial position to complete imaging of the patient KR. After this, the CPU 70 transmits the final tomographic medical image 50f to the image server 14. The volume data 52 is obtained as one diagnosis, and transmitted to the image server 14.

The CPU 90 in the image server 14 receives the tomographic medical images 50 output serially by the computed tomography apparatus 12, and then inputs the tomographic medical images 50 into the site recognition unit 90a. The site recognition unit 90a calculates characteristic values of the tomographic medical images 50 and the like, and recognizes a site recorded in the tomographic medical images 50. The site recognition unit 90a records the site information 56e of the site in the tag region 56, to determine the site information 56e in a provisional manner.

The CPU 90 after the image recognition in the site recognition unit 90a refers to the image end flag 56d of the tomographic medical images 50 in the state of provisionally determining the site information 56e. The CPU 90 checks whether the transfer of the tomographic medical images 50 is completed. If the transfer of the tomographic medical images 50 is found incomplete, the CPU 90 waits for further transfer. At each time of reception of the tomographic medical images 50, provisional determination of the site information 56e is carried out.

When the transfer of the tomographic medical images 50 is found completed, the CPU 90 refers to the imaging number 56c of the tomographic medical images 50, and aligns the tomographic medical images 50 in the sequence of the imaging number 56c. It is likely that the sequence of the tomographic medical images 50 transferred by the computed tomography apparatus 12 may change incidentally according to a status of loading of the LAN 20 or the CPU 90. However, the tomographic medical images 50 are aligned for the purpose of rearrangement or correction of the sequence after a temporary change generated in the transfer.

It is likely that the final tomographic medical image 50f is transferred incidentally earlier than a final step. In consideration of this, the CPU 90 checks the imaging number 56c of one of the tomographic medical images 50 with a setting of one (1) as the image end flag 56d in response to its reception. The CPU 90, if the checked value of the imaging number 56c coincides with the number of the tomographic medical images 50 being received, determines that the transfer of the tomographic medical images 50 is completed. The use of the imaging number 56c and the image end flag 56d for check is effective in correctly verifying the full transfer of the tomographic medical images 50 even with a difference between the number of images of request and the number of actually recorded images. Furthermore, there is an advantage in unnecessity in designation of the number of images at every time of sending the request.

After the rearrangement of the tomographic medical images 50, the CPU 90 refers to the site information 56e of the tomographic medical images 50 of the provisional determination. The site information 56e of a first one of the tomographic medical images 50 is checked by referring to the site information 56e of one of the tomographic medical images 50 preceding to the first and the site information 56e of a second one of the tomographic medical images 50 next to the first, and discovers any error in the recognition of the site information 56e, to determine the site information 56e in a final step. In short, the final determination is the second recognition according to the invention. The provisional determination is the first recognition of the invention.

The CPU 90 having discovered an error corrects the site information 56e to change into an exact site. For example, sites of the site information 56e of the tomographic medical images 50 are in a sequence of the chest, pelvic bone, chest, abdomen and abdomen. The order of the pelvic bone between the items of the chest is estimated as incorrect apparently in view of the image recognition. Then the site information 56e of one of the tomographic medical images 50 misread as pelvic bone is changed to the chest. Precision in the image recognition of the tomographic medical images 50 can be high, owing to the double recognition with the provisional determination and the final determination.

The CPU 90 after the final determination of the site information 56e stores the volume data 52 in the hard disk drive 92 as a result of the diagnosis of the patient KR. The volume data 52 in the hard disk drive 72 of the computed tomography apparatus 12 as a source of the transfer can be deleted from the hard disk drive 72 in response to reception of a message of completion of the storage generated by the image server 14.

In the above embodiments, the imaging number 56c is used as sequence information. However, sequence information of the invention may be letters, numbers or other alphanumeric information for the purpose of expressing an order of a medical image. In the above embodiments, image end information is the image end flag 56d. Alternatively, image end information may be alphanumeric expression as words, the total number of medical images being acquired. In the above embodiments, the sequence information and image end information are assigned to medical images as metadata. However, attribute information of each of the types may be recorded in a separate data file, which can be transferred with medical images in a combined manner.

In the above embodiments, transfer of the first of the tomographic medical images 50 is simultaneous with the imaging of the second of the tomographic medical images 50. Image data of the tomographic medical images 50 is a data stream. However, it is possible to store the tomographic medical images 50 in the hard disk drive 72, and to transfer the tomographic medical images 50 discretely one after another after completion of imaging of all the tomographic medical images 50. Also, the start of the site recognition is not limited to the above embodiment. For example, the site recognition may be started after reception of all the tomographic medical images 50.

In the above embodiments, a medical image processor of the invention is the image server 14. However, other components in a system may operate as a medical image processor or manager in which transmitted images are received. In the above embodiments, the first and second tasks of the processor are the provisional and final determination of the site information 56e. However, first and second tasks may be other processing in a processor, for example, halftone conversion or other image processing, compression, or the like. In the above embodiments, sites targeted by the site recognition unit 90a are the head, chest, abdomen, pelvic bone, leg and the like. However, internal body parts or organs can be included in sites of the recognition, for example, brain, heart, lungs, liver, stomach and the like.

In the above embodiment, a medical imaging apparatus of the invention is the computed tomography apparatus 12. However, medical imaging apparatuses of any of various modalities may be used, for example, MRI (magnetic resonance imaging) apparatus, PET (positron emission tomography) apparatus or the like for retrieving plural medical images at one time of diagnosis. Also, a medical imaging apparatus may be a scanner which may optically read medical images from films or other recording medium for digitizing.

In the embodiment, the medical network system 10 is used in one hospital or department of the medical facilities. However, the medical network system 10 may be used in plural hospitals or departments of medical facilities in a combined set by communication lines.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A medical network system including a medical imaging apparatus for imaging a body to acquire plural medical images of a consecutive series, and a medical image processor for processing an image group of said medical images transferred from said medical imaging apparatus by network transmission, said medical network system comprising:
said medical imaging apparatus including:
a sequence information generator for generating sequence information of a sequence of imaging of each of said medical images;
an image end information generator for generating image end information representing a final medical image acquired finally in said image group;
a transmitter for transferring said medical images to said medical image processor;
said medical image processor including a checker for checking said sequence information and said image end information and for verifying completion of transfer of said medical images in said image group.

2. A medical network system as defined in claim 1, wherein said medical image processor performs a first task related to said medical images discretely from one another, and then a second task related to said medical images in total after said checker verifies said completion of said transfer of said medical images.

3. A medical network system as defined in claim 2, wherein said medical image processor further includes a site recognition unit for recognizing a site of said body recorded in each of said medical images by image recognition, to create site information of said medical images.

4. A medical network system as defined in claim 3, wherein said site recognition unit recognizes said medical images discretely from one another, and recognizes said medical images in total after said checker verifies said completion of said transfer of said medical images;
wherein in said discrete recognition, said site information of said medical images is determined in provisional determination by image analysis of said medical images;
in said recognition in total, said site information is checked, to determine new site information of said medical images in final determination by considering an error in recognition.

5. A medical network system as defined in claim 1, wherein said checker identifies said final medical image according to said image end information, and determines said completion of said transfer of said medical images in said image group when a number of said final medical image coincides with a number of said medical images by referring to said sequence information of said final medical image.

6. A medical network system as defined in claim 1, wherein said sequence information is metadata assigned to respectively said medical images, and said image end information is metadata assigned to said final medical image.

7. A medical network system as defined in claim 1, wherein said sequence information generator sequentially assigns said sequence information to said medical images being acquired, and said transmitter transfers said medical images to said medical image processor sequentially after assignment of said sequence information.

8. A medical network system as defined in claim 1, wherein said medical images are tomographic.

9. A medical imaging apparatus for imaging a body to acquire an image group constituted by plural medical images of a consecutive series, comprising:
a sequence information generator for generating sequence information of a sequence of imaging of each of said medical images; and
an image end information generator for generating image end information representing a final medical image acquired finally in said image group.

10. A medical imaging apparatus as defined in claim 9, further comprising a transmitter for transferring said medical images, said sequence information and said image end information;
wherein said sequence information generator sequentially assigns said sequence information to said medical images being acquired, and said transmitter transfers said medical images sequentially after assignment of said sequence information;
said sequence information and said image end information are used for verifying completion of transfer of said medical images in said image group after transfer of said medical images.

11. A medical image processor for processing an image group of medical images transferred by network transmission, said medical images being acquired in a consecutive series, comprising:
a checker, supplied with sequence information of a sequence of imaging of each of said medical images of said image group and image end information representing a final medical image acquired finally in said image group, for verifying completion of transfer of said medical images in said image group by checking said sequence information and said image end information.

12. A medical image processor as defined in claim 11, further comprising a site recognition unit for recognizing a site of said body recorded in each of said medical images by image recognition, to create site information of said medical images.

13. A medical image processor as defined in claim 12, wherein said site recognition unit recognizes said medical images discretely from one another, and recognizes said medical images in total after said checker verifies said completion of said transfer of said medical images;
in said discrete recognition, said site information of said medical images is determined in provisional determination by image analysis of said medical images;
in said recognition in total, said site information is checked, to determine new site information of said medical images in final determination by considering an error in recognition.

14. A medical image processor as defined in claim 11, wherein said checker identifies said final medical image according to said image end information, and determines said completion of said transfer of said medical images in said image group when a number of said final medical image coincides with a number of said medical images by referring to said sequence information of said final medical image.

15. A medical image processing method of image processing of an image group of plural medical images received discretely by network transmission, said medical images being acquired in a consecutive series, said medical image processing method comprising steps of:
recognizing said medical images discretely from one another by image recognition;
verifying completion of transfer of said medical images in said image group according to sequence information of a sequence of imaging of each of said medical images, and image end information representing a final medical image acquired finally in said image group; and
recognizing said medical images in total by image recognition after said completion of said transfer of said medical images is verified.

16. A medical image processing method as defined in claim 15, wherein in said discretely recognizing step, a site of a body recorded in said medical images is recognized to generate site information of said medical images.
